(19) United States
(12) Patent Application Publication
(10) Pub. No.: US 2007/0282302 A1
(43) Pub. Date: Dec. 6, 2007

(54) Stent Positioning Using Inflation Tube

(75) Inventors: Oded Wachsman, Tel-Aviv (IL); Isrrael Chermoni, Haifa (IL)

Correspondence Address: WOLF, BLOCK, SCHORR & SOLIS-COHEN LLP, 250 PARK AVENUE, NEW YORK, NY, 10177, US

(73) Assignee: F.D. Cardio LTD, Tel-Aviv (IL)

(21) Appl. No.: 10/595,986

(22) PCT Filed: May 27, 2004
(86) PCT No.: PCT/IL04/00456

§ 371 Date: Jul. 11, 2007

(51) Int. Cl. A61M 25/10

(52) U.S. Cl. 604/509; 604/103.02; 623/1.11

(57) ABSTRACT

A catheter adapted for performing a task at a location inside a lumen, the catheter comprising: a) an outer sheath; b) a balloon capable of inflating inside the lumen when the catheter reaches the location; and c) a balloon inflation tube, which is attached to the balloon and carries a fluid which causes the inflating of the balloon, said balloon inflation tube running through the outer sheath, movable relative to the outer sheath, and stiff enough so that it can be used to push and pull the balloon relative to the outer sheath.

### RELATED APPLICATIONS

This application claims priority from and is a continuation-in-part of PCT application PCT/IL03/00995 filed on Nov. 25, 2003, which designates the United States, and is also a continuation-in-part of U.S. application Ser. No. 10/303,064, filed on Nov. 25, 2002, the disclosures of which are incorporated herein by reference. This application also incorporates by reference a patent application titled “Catheter Drive,” agent's file reference 378/04070, filed on May 27, 2004, the same day as the current application, at the United States Patent and Trademark Office.

### FIELD OF THE INVENTION

The field of the invention is catheters, particularly for positioning and advancing stents and other medical devices in blood vessels and other lumens of the body.

### BACKGROUND OF THE INVENTION

Blood vessels can suffer from various diseases, in particular arteriosclerosis, in which obstructions form in a lumen of a blood vessel, narrowing or clogging it. Emboli can also cause clogging of blood vessels. A common treatment method for narrowing is inserting a catheter with a balloon at its end to a clogged portion of the blood vessel, inflating the balloon and possibly leaving a stent at the clogged location, to keep the blood vessel open.

Precise positioning of the stent in the blood vessel is important. For example, stents are often placed over plaque in arteries. Placing the stent at a wrong position, for example not directly over the plaque, can increase the chance of complications such as stent thrombosis and restenosis. Precise positioning also allows a stent with the right length to just cover the plaque. It is also important, in the case of plaque located near a bifurcation in an artery, not to position the stent so that it will block the ostium of the artery in the bifurcation. However, it is often difficult for physicians to precisely control the position of a catheter, even when imaging techniques make it possible to tell exactly where the stent is, relative to the plaque. This difficulty is due to the friction between the catheter and the blood vessel. A certain force is needed to get the catheter to start moving, but once it starts moving, the catheter might jump a finite distance. Typically, the friction is dominated by the body of the catheter, which can be a meter or more in length, and not by the balloon and the stent, which tend to have relatively little friction with the blood vessel wall when the balloon is not inflated.

In some implementations, a guide wire is brought to the narrowed location, and then the catheter is pushed over the guide wire. If the catheter is soft, helping it to negotiate curves, it may be difficult to convey the pushing force along the catheter from outside the body to the tip. If the catheter is stiffer, it may be less able to negotiate sharp curves. In either case, excessive pushing may damage the blood vessels.

One solution suggested in the art is providing a catheter with varying levels of stiffness along its length—stiff at its proximal end and soft at its distal end.

Catheters with a balloon at the distal end typically have a separate lumen for an inflation tube, made of flexible plastic or metal for example, to inflate the balloon. The inflation tube is typically part of and inside a sheath which is stiff enough to push through the blood vessel.

### SUMMARY OF THE INVENTION

An aspect of some embodiments of the invention concerns a catheter with a balloon, for example for expanding a stent. In an embodiment, the catheter comprises a relatively stiff inflation tube inside an outer sheath, and the inflation tube can move relative to the outer sheath. The catheter as a whole is used for coarse positioning of the balloon in a blood vessel or other lumen, as in conventional catheters. For example, the catheter is pushed and sometimes twisted by hand, while its progress is followed by using medical imaging device such as a fluoroscope. For fine adjustment in the position of the balloon, the inflation tube is used to push or pull the balloon, while keeping the outer sheath in place relative to the blood vessel wall. Because there is less friction between the inflation tube and the outer sheath than there is between the outer sheath and the blood vessel, there is better control on the positioning of the balloon and the stent and there is little or no jumping when using the inflation tube to move the balloon, and the balloon may be positioned accurately. The inflation tube is stiff enough to push or pull the balloon by itself. In addition, the stiffness of the inflation tube helps make the catheter as a whole stiff enough for the initial coarse positioning of the balloon. Once the balloon is accurately positioned, it is used to expand a stent, or to perform another task.

An aspect of some embodiments of the invention concerns a catheter with a balloon and an inflation tube, in which the inflation tube comprises two tubes: a relatively stiff inner inflation tube surrounded by a relatively flexible outer inflation tube. The inner inflation tube can be manipulated relative to the outer inflation tube from outside the body. Normally, both the inner and outer inflation tubes extend to the distal end of the catheter. When it is desired for the catheter to have a more flexible end, for example in order to negotiate sharp turns without damaging the blood vessel, the inner inflation tube is drawn back from the distal end of the catheter. This leaves an end portion of the outer inflation tube empty, and makes the end portion of the catheter more flexible than it would be with the inner inflation tube inside. When it is desired for the catheter to have a stiffer end, for example in order to push past a narrow region in a blood vessel, then the inner inflation tube is pushed to the distal end of the catheter. The relatively stiff inner inflation tube then stiffens the end portion of the catheter.

Optionally, both of these aspects may be present in an embodiment of the invention, in which there are three concentric tubes, an outer sheath, a relatively flexible outer inflation tube and a relatively stiff inner inflation tube, which may be moved relative to each other. Optionally, either or both of these features may be combined with the hydraulic mechanism for advancing the catheter described in PCT/IL03/00995. In that case, there are as many as four concentric tubes: an outer sheath, an inner sheath which comprises the hydraulic mechanism, an outer inflation tube inside the inner sheath, and an inner inflation tube. The outer inflation tube, or the inflation tube if there is only one tube, need not move relative to the inner sheath. The physician then has at his disposal three different means to position the balloon: 1) moving the outer sheath, i.e. the whole catheter, as in conventional catheters; 2) using the hydraulic mechanism to move the inner sheath, together with the inflation tube and the balloon; and 3) using the inflation tube to move the inner sheath and the balloon. Once the balloon is in the desired position, it is inflated, for example in order to expand a stent which surrounds the balloon, and once the stent is in place, or another therapeutic or diagnostic task has been accomplished, the balloon is deflated and the catheter is withdrawn.

There is thus provided, according to an exemplary embodiment of the invention, a catheter adapted for performing a task at a location inside a lumen, the catheter comprising:
a) an outer sheath;
b) a balloon capable of inflating inside the lumen when the catheter reaches the location;

and
c) a balloon inflation tube, which is attached to the balloon and carries a fluid which causes the inflating of the balloon, said balloon inflation tube running through the outer sheath, movable relative to the outer sheath, and stiff enough so that it can be used to push and pull the balloon relative to the outer sheath.

In an embodiment of the invention, the inflation tube comprises:
a) a relatively flexible outer balloon inflation tube with a lumen, extending substantially to the tip of the catheter; and
b) a relatively stiff inner inflation tube element, which runs through the lumen of the outer balloon inflation tube and is movable with respect to the outer balloon inflation tube;
   whereby moving the inner inflation tube element back from the tip of the catheter makes a distal portion of the catheter substantially more flexible than when the inner inflation tube extends to the tip of the catheter.

Optionally, the inner inflation tube element has a lumen which carries the fluid which causes the inflating of the balloon.

In an embodiment of the invention, the catheter includes a propulsion compartment located proximal to the balloon, the propulsion compartment comprising an outer tube and an inner tube, said tubes being concentric, wherein one of said outer tube and inner tube can slidingly move in relation to the other of said outer tube and inner tube in response to a pressure exerted thereon by a fluid introduced into one or both of said outer tube and inner tube.

Optionally, one of said outer tube and inner tube is the outer sheath, and the balloon inflation tube runs through and is attached to the other of said outer tube and inner tube.

Optionally, the outer tube is the outer sheath.

Alternatively, the inner tube is the outer sheath.

Optionally, the task comprises dilating the lumen.

Optionally, the lumen is inside the body.

Optionally, the lumen is a blood vessel.

Optionally, the task comprises placing a stent.

Optionally, the balloon inflation tube comprises stainless steel.

Alternatively, the balloon inflation tube comprises NiTi.

Optionally, the balloon comprises plastic.

Alternatively or additionally, the balloon comprises a polymer.

In an embodiment of the invention, the catheter is adapted for using a guide wire.

Optionally, the catheter is adapted for using an “over the wire” guide wire.

Alternatively or additionally, the catheter is adapted for using a “rapid exchange” guide wire.

There is further provided, in accordance with an exemplary embodiment of the invention, a method of positioning a balloon of a balloon catheter in a lumen, the method comprising:
a) positioning the balloon approximately; and then
b) fine adjusting the position of the balloon, said fine adjusting comprising moving an inflation tube of the balloon catheter relative to an outer sheath of said catheter, by manually manipulating said inflation tube.

Optionally, moving the inflation tube relative to the outer sheath comprises moving the inflation tube while keeping the outer sheath stationary with respect to the lumen.

Optionally, positioning the balloon approximately comprises moving the entire catheter through the lumen.

Alternatively or additionally, positioning the balloon approximately comprises using hydraulic force.

Optionally, fine adjusting also comprises using hydraulic force to move the balloon, while keeping the outer sheath of the catheter stationary with respect to the lumen.

There is further provided, in accordance with an exemplary embodiment of the invention, a method of manipulating a balloon catheter through a lumen comprising both sharply curved portions and partially obstructed straight portions, the method comprising:
a) arranging a moveable stiffening element to extend substantially to the tip of the catheter, when manipulating the tip of the catheter through the partially obstructed straight portions; and
b) arranging the moveable stiffening element to be withdrawn some distance back from the tip of the catheter, when manipulating the tip of the catheter past the sharply curved portions.

Optionally, the stiffening element is located inside a balloon inflation tube of said catheter.

Alternatively, the stiffening element comprises a balloon and a balloon inflation tube of said catheter, and arranging the stiffening element to be withdrawn some distance back comprises withdrawing the balloon into an outer sheath of said catheter.

### BRIEF DESCRIPTION OF THE DRAWINGS

Exemplary, non-limiting embodiments of the invention will be described below, with reference to the following drawings, which are not generally drawn to scale. The same elements are marked with the same or similar reference numbers in different drawings.

FIG. 1 is a schematic side cross-sectional view of a catheter, according to an exemplary embodiment of the invention;

FIGS. 2A through 2E are a time series of schematic side views of a catheter inside a side cross-sectional view of a blood vessel, showing how the catheter sets a stent, according to the embodiment of the invention shown in FIG. 1;

FIG. 3A is a schematic side cross-sectional view of a catheter, according to another exemplary embodiment of the invention;

FIG. 3B is a schematic side cross-sectional view of a catheter, according to another exemplary embodiment of the invention;

FIGS. 4A and 4B are schematic side cross-sectional views showing two different states of a catheter, according to another exemplary embodiment of the invention;

FIGS. 5A and 5B are schematic side cross-sectional views of a catheter inside a blood vessel, illustrating uses of the two states of the catheter shown in FIGS. 4A and 4B; and

FIG. 6 is a schematic side cross-sectional view of a catheter according to another exemplary embodiment of the invention.

### DETAILED DESCRIPTION OF EXEMPLARY EMBODIMENTS

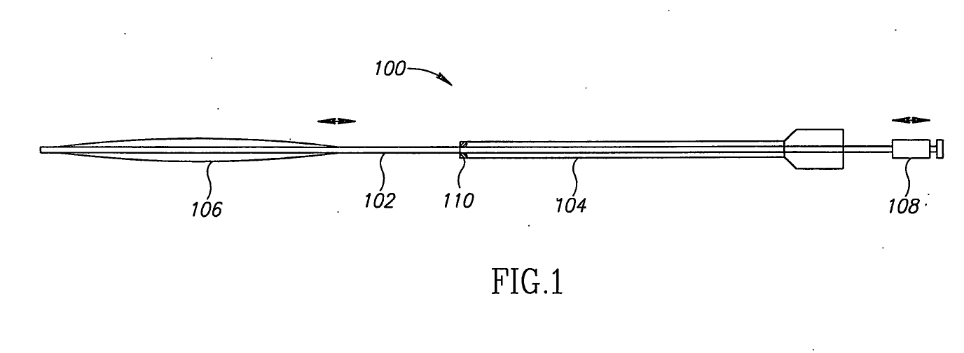

FIG. 1 shows an intravascular balloon catheter 100, in accordance with an embodiment of the invention, comprising an inflation tube 102 surrounded by an outer sheath 104. There is a balloon 106 at the end of inflation tube 102, which is stiff enough so that the inflation tube can be used to push the balloon relative to the rest of the catheter, for fine adjustment in positioning the balloon.

FIG. 1 is not necessarily drawn to scale, and in particular, the outer sheath and inflation tube of catheter 100 are typically very much longer, relative to their diameter, than shown in FIG. 1. This is also true of the catheters in all the other drawings. Also, in FIG. 1 and in the other drawings, balloon 106 is shown in a somewhat inflated state, for clarity. In practice the balloon will be in a collapsed state, fitting fairly closely around the inflation tube, until it is in position and ready for inflating.

An inflation tube manipulator 108, outside the body, allows the inflation tube, together with the balloon, to be moved back and forth axially with respect to the outer sheath. Optionally, inflation tube manipulator 108 also includes, or is connected to, an indeflator, i.e. an element for pumping a fluid such as saline solution into the balloon, to inflate it. If manipulator 108 includes a reservoir and plunger for pumping fluid into balloon 106, it would preferably be bigger, relative to balloon 106, than shown in FIG. 1, and this is true of inflation tube manipulators appearing in the other drawings as well. One or more optional seals 110 may be located at the distal end of the outer sheath, as shown in the drawing, or outside the body at the proximal end of the outer sheath, or anywhere in between. Seal 110 prevents blood from leaking out of the body, and prevents air and other material from getting into the blood vessel, through the catheter. Alternatively, inflation tube 102 fits closely enough to the inner surface of outer sheath 104 that there is no need for a special seal. Optionally, there is also a guide wire, not shown in the drawing, which may use either a “rapid exchange” (monorail) system, as described, for example, in U.S. Pat. No 4,748,982 to Horzewski and Yock, the disclosure of which is incorporated herein by reference, or an “over the wire” system.

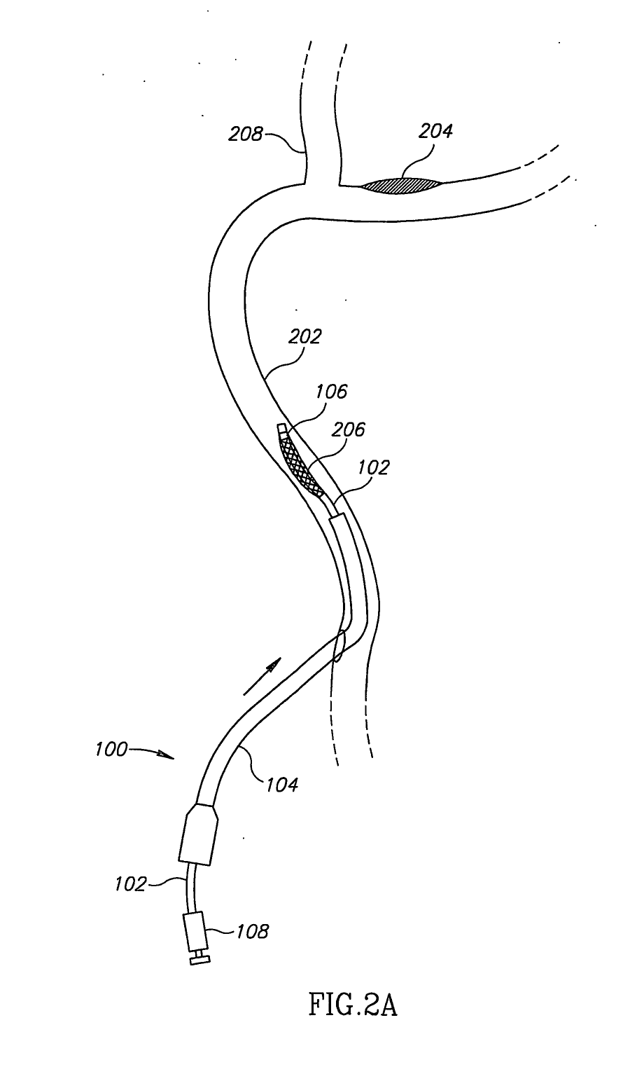

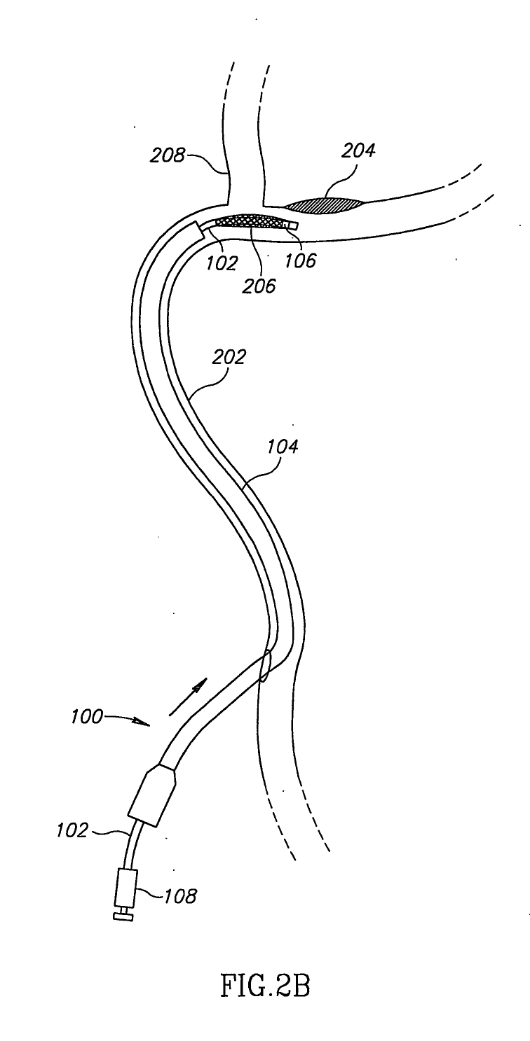

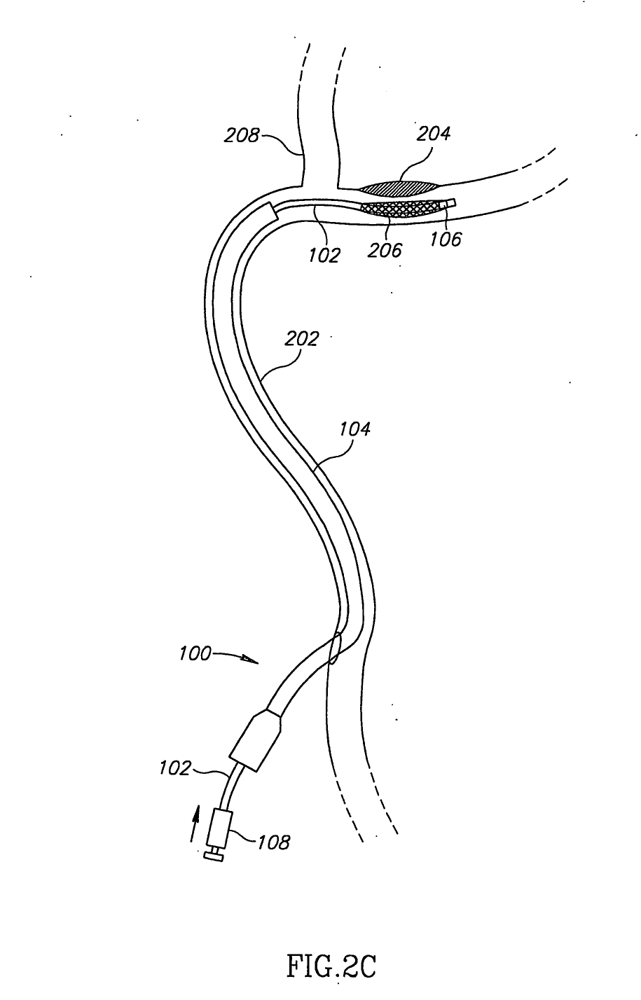

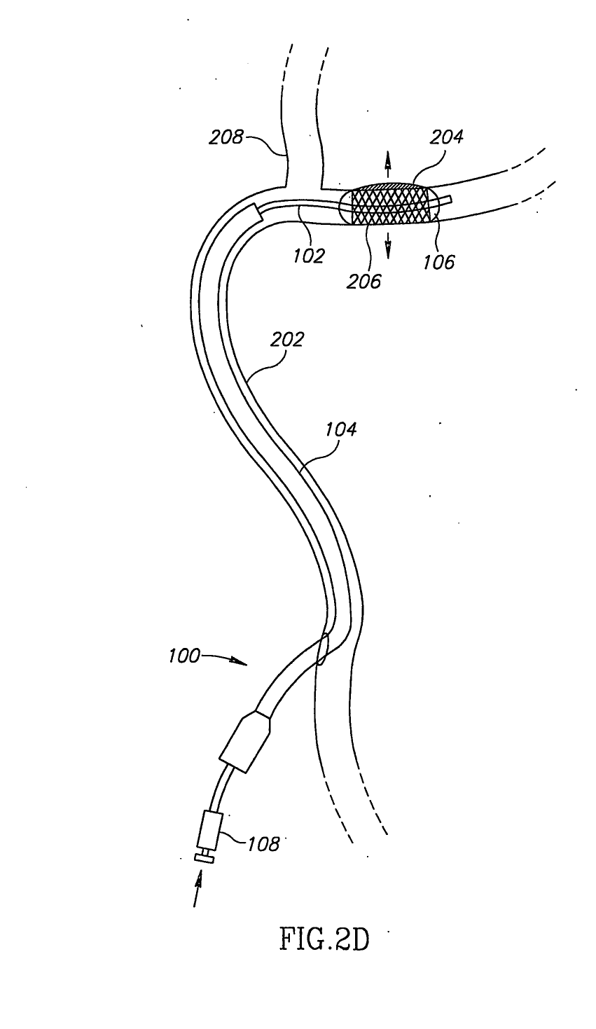

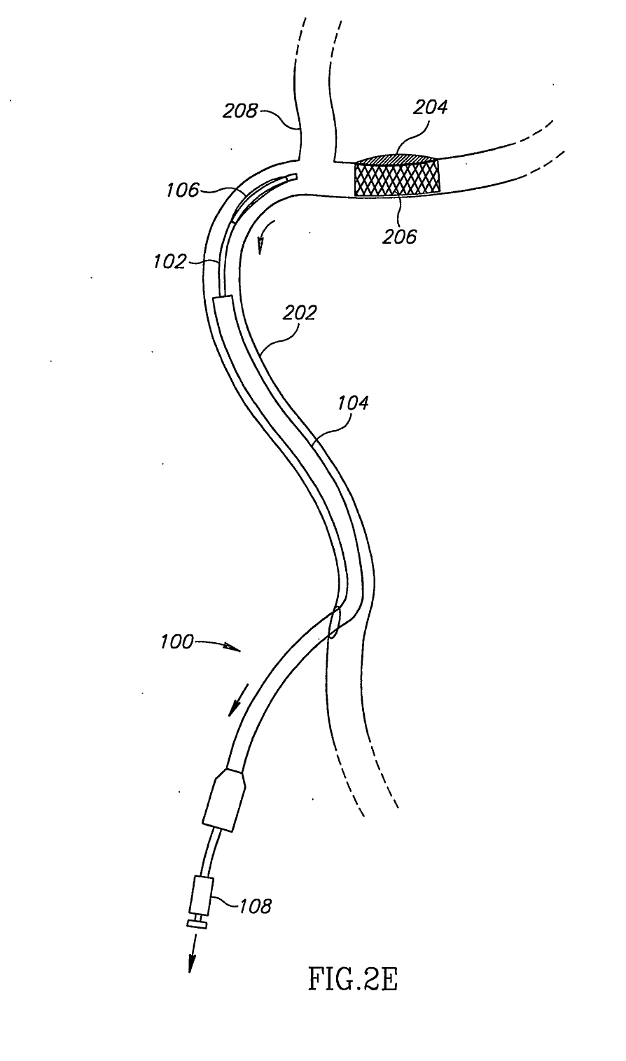

The balloon catheter may be used, for example, to place a stent in an artery, as shown in FIGS. 2A-2E, or to enlarge the lumen of a partially obstructed artery by angioplasty or arthrectomy, or for any other therapeutic or diagnostic purpose for which conventional balloon catheters are used. Such applications include thermal ablation of tissue (using RF, laser, or ohmic heating for example), cryotherapy, photodynamic therapy, drug delivery, dilation of various lumens in the body (including, for example, esophagus, bile duct, urethra, Fallopian tube, heart valve, tear duct, and carpal tunnel), positioning devices for radiation therapy or for imaging (for example using ultrasound), occluding or sealing openings, and delivering endovascular grafts. Balloon catheter 100 is particularly useful for applications in which precise positioning of the balloon is important.

In FIG. 2A, outer sheath 104 of catheter 100, together with inflation tube 102, and balloon 106 with a stent 206 fitted around it, is initially manipulated through a blood vessel 202, similarly to a conventional intravascular catheter, until the balloon and stent are approximately in the desired position in the blood vessel. If the catheter includes a guide wire, not shown in FIG. 2A, then the guide wire is optionally pushed first into position through the blood vessel, followed by the catheter. In FIG. 2B, balloon 106 has reached a location close to plaque 204 in the wall of blood vessel 202. It is desired to place stent 206 precisely over plaque 204, but not to have the stent block the opening of a blood vessel 208 which branches off blood vessel 202 near plaque 204. In FIG. 2C, outer sheath 104 remains in place relative to blood vessel 202, and manipulator 108 is used to move inflation tube 102 and balloon 106 toward lesion 204. Because outer sheath 104 and inflation tube 102 are optionally designed to have much less friction and stiction moving against each other than the outer sheath has when moving against the blood vessel wall, it is potentially easier to precisely position the balloon in this way, than it would be by moving the whole catheter in the blood vessel. Finally, in FIG. 2D, balloon 106 is in the precise position desired, centered at lesion 204, and past the opening of blood vessel 208. Balloon 106 is expanded, by pumping fluid into it, under pressure, through inflation tube 102. The expanding balloon causes stent 206 to expand, and anchors it in place. In FIG. 2E, balloon 106 has been deflated, by releasing the pressure through inflation tube 102, and catheter 100 is being withdrawn, leaving stent 206 in place, precisely covering lesion 204, but not blocking the opening of blood vessel 208.

Optionally, balloon catheter 100 performs any other therapeutic or diagnostic task, including any of those listed above, once balloon 106 is in position, instead of or in addition to placing the stent. Similar balloon catheters, of appropriate dimensions, are optionally used in lumens other than blood vessels.

Inflation tube 102 is optionally made of stainless steel, nickel-titanium (Nitinol), or other biocompatible materials with the right mechanical properties, including plastics or polymers. The inflation tube is preferably stiff enough so that it can be used to push the balloon for fine positioning, but flexible enough so that it can bend with the catheter in going around sharp curves in blood vessels. Also, outer sheath 104, in combination with inflation tube 102, is stiff enough to enable the catheter to be manipulated through the blood vessel for coarse positioning, but flexible enough to allow the catheter to follow turns in the blood vessel.

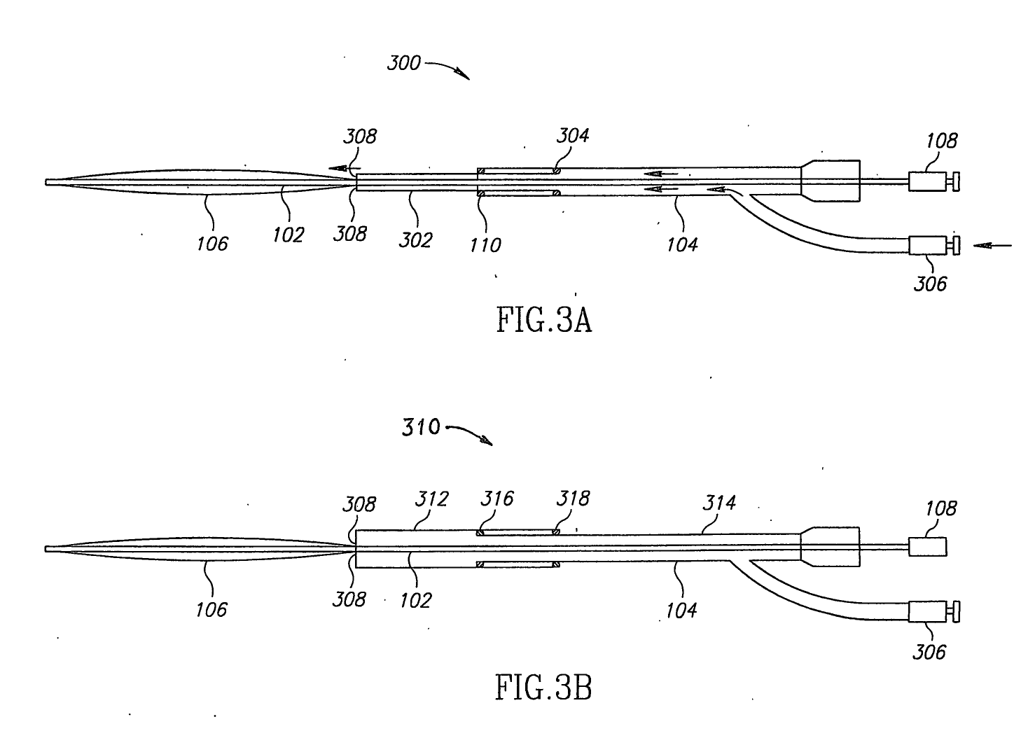

FIG. 3A shows a catheter 300, similar to catheter 100 in FIG. 1, but with the additional capability of using hydraulic force to move the balloon, as described in PCT/IL03/00995. Like catheter 100, catheter 300 has a balloon 106 attached to an inflation tube 102, a manipulator 108 at the proximal end of inflation tube 102, and an outer sheath 104. Catheter 300 also has an inner sheath 302, attached to the balloon. Inner sheath 302, which is moveable relative to outer sheath 104, surrounds inflation tube 102 and fits inside outer sheath 104. Inner sheath 302 does not extend over the whole length of the catheter, but ends at a seal 304, before the proximal end of outer sheath 104. Seal 304 can move smoothly along the inside surface of outer sheath 104, but seal 304 does not allow fluid to pass through it. At its distal end, inner sheath 302 optionally ends at a surface 308, which extends radially from the inner surface of inner sheath 302 to the outer surface of inflation tube 102.

The portion of outer sheath 104 proximal to seal 304 is filled with saline solution or another relatively incompressible fluid, and there is an indeflator 306, or a similar hydraulic displacement element such as a plunger or a flexible bulb, located outside the body at the proximal end of outer sheath 104. When indeflator 306 is compressed, pressure builds up in the fluid, which exerts an unbalanced longitudinal force on surface 308. This force causes inner sheath 302 to move, together with balloon 106 which is attached to inner sheath 302. This forward motion of inner sheath 302 increases the volume of fluid again, relieving the build up in pressure. Realistically, indeflator 306 would preferably be larger in volume, relative to the volume of inner sheath 302, than shown in FIG. 3A, and this is true of the indeflators in other drawings as well.

Indeflator 306, like manipulator 108, is optionally used only for fine positioning of balloon 106, after coarse positioning the balloon by manipulating the entire catheter through the blood vessel. In this case, inner sheath 302 is optionally rather short, only as long as the greatest distance needed for fine positioning. Also in this case, there is optionally no inner sheath 302 at all, and seal 304 is attached directly to inflation tube 102. But using an inner sheath, with surface 308 greater in area than seal 304, has the potential advantage that most of the force is applied to surface 308, which is close to the location of the balloon, rather than to seal 304, which is removed some distance from the balloon.

Alternatively, indeflator 306 is used not just for fine positioning, but for the initial coarse positioning of the balloon as well, instead of or in addition to manipulating outer sheath 104, as in a conventional catheter, for initial positioning. In this case, inner sheath 302 is optionally almost as long as outer sheath 104, and seal 304 is optionally located near the proximal end of outer sheath 104 initially. Particularly in this case, surface 308 is preferably much greater in area than seal 304, so that the hydraulic force is exerted mostly on surface 308, near the balloon, and not on seal 304, which may be very far from the balloon.

Optionally, whether or not indeflator 306 is used for initial coarse positioning of the balloon, inner sheath 302 makes a sufficiently close fit to the inside of outer sheath 104 that there is no need for seal 304 at all, and almost all of the hydraulic force is applied to surface 308, near the balloon. Alternatively, there is a seal attached to the distal end of outer sheath 104 which prevents fluid from leaking out, instead of or in addition to seal 304.

Depending on the shape and texture of the inner wall of the blood vessel adjacent to the balloon, either indeflator 306 or manipulator 108 may be more effective for fine positioning of the balloon, or manipulating the catheter as a whole may even be more effective. Alternatively, a combination of two of these methods of moving the balloon, or all three, may be more effective than any one of them. A potential advantage of catheter 300 is that the physician has a choice of these three options when positioning the balloon.

Optionally, indeflator 306 can only exert a force which moves balloon 106 in a distal direction. Alternatively, indeflator 306 is reversible, and can be used both to pull and to push on surface 308 and seal 304, moving balloon 106 in either a distal or a proximal direction. In the latter case, indeflator 306 can be used to adjust the position of the balloon by pulling it back if it is inadvertently pushed too far. In the former case, only manipulator 108, or the catheter as a whole, can be used to pull the balloon back.

FIG. 3B shows a catheter 310 which is similar to catheter 300, but with what used to be the “inner” sheath now located outside the outer sheath. To avoid misleading terminology, the former “inner” sheath will be referred to as the “outermost sheath” in FIG. 3B, since it is outside the outer sheath. The outer sheath will still be called the “outer sheath” because it is outside the inflation tube, even though it is now inside the outermost sheath. Thus, in catheter 310, there is an outermost sheath 312 which is attached to inflation tube 102 and balloon 106, and there is a surface 308 at the distal end of outermost sheath 312. There is an outer sheath 314, between outermost sheath 312 and inflation tube 102, which does not extend to the distal end of outermost sheath 312. Outermost sheath 312 does not extend to the proximal end of outer sheath 314. Seals 316 and 318, respectively attached to the proximal end of outermost sheath 312 and the distal end of outer sheath 314, allow the outer and outermost sheaths to slide along each other, but do not allow fluid to leak out between them. Optionally, only one of these seals is present, or the outer and outermost sheaths fit closely enough together so that there is no need for either seal. A manipulator 108, at the proximal end of inflation tube 102, allows inflation tube 102, together with outermost sheath 312 and balloon 106, to be pushed or pulled relative to outer sheath 314, for fine positioning of the balloon. Outer sheath 314, and a distal portion of outermost sheath 312 up to seal 318, are filled with a relatively incompressible fluid, such as a saline solution, which is connected to an indeflator 306 or similar hydraulic displacement element located outside the body. The indeflator exerts a hydraulic force on surface 308, causing outermost sheath 312, together with inflation tube 102 and balloon 106, to move relative to outer sheath 314. Depending on how long outermost sheath 312 is, indeflator 306 is used either only for fine positioning of the balloon, or for both coarse and fine positioning of the balloon.

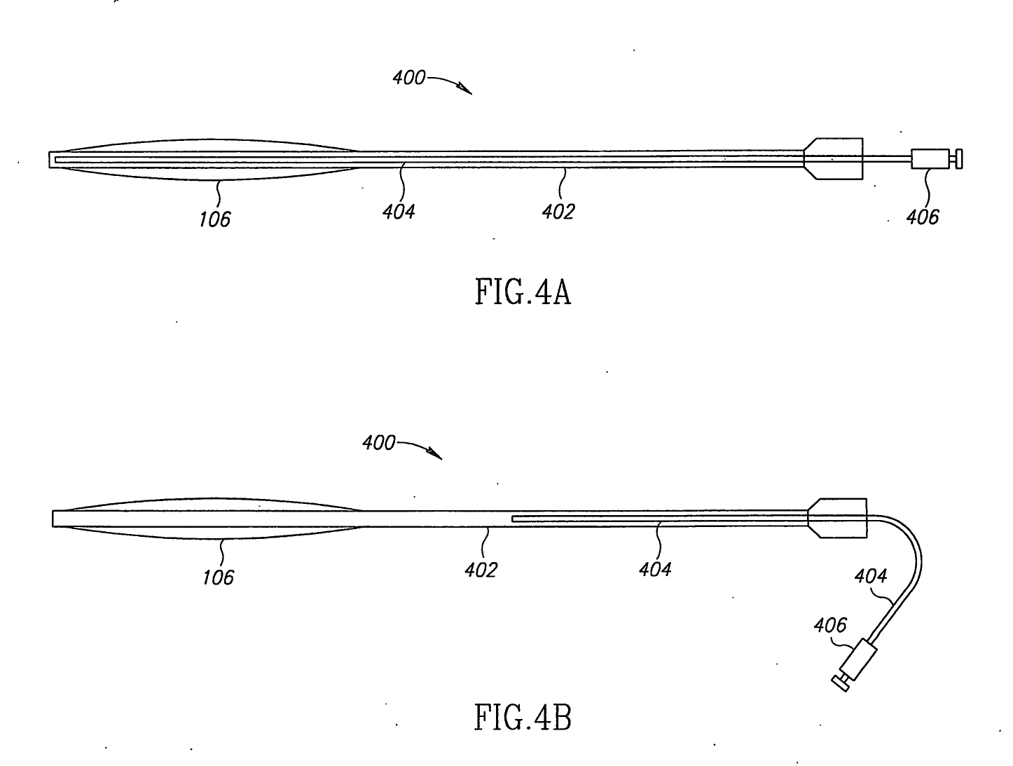

FIG. 4A shows a catheter 400, the distal portion of which can be adjusted in stiffness. Catheter 400 has a balloon 106 attached to a relatively flexible outer balloon inflation tube 402. An inner inflation tube 404 is relatively stiffer than outer inflation tube 402, or at least the two tubes together are significantly stiffer than the outer inflation tube by itself. The inner inflation tube can move relative to the outer inflation tube. A manipulator 406 is attached to the proximal end of inner inflation tube 404, outside the body, and can be used to move inner inflation tube 404 relative to outer inflation tube 402. Optionally, manipulator 406 also includes, or is connected to, an indeflator or similar element for pumping a fluid such as saline solution into the balloon, to inflate it.

In FIG. 4A, inner inflation tube 404 is pushed as far as it will go toward the distal end of outer inflation tube 402, at or past the end of balloon 106. In this state, the distal portion of catheter 400, out to the tip, is relatively stiff. In FIG. 4B, inner inflation tube 404 is shown withdrawn some distance back from the distal end of outer inflation tube 402. In this state, the distal portion of catheter 400, back to the end of inner inflation tube 404, is substantially more flexible than it was in FIG. 4A.

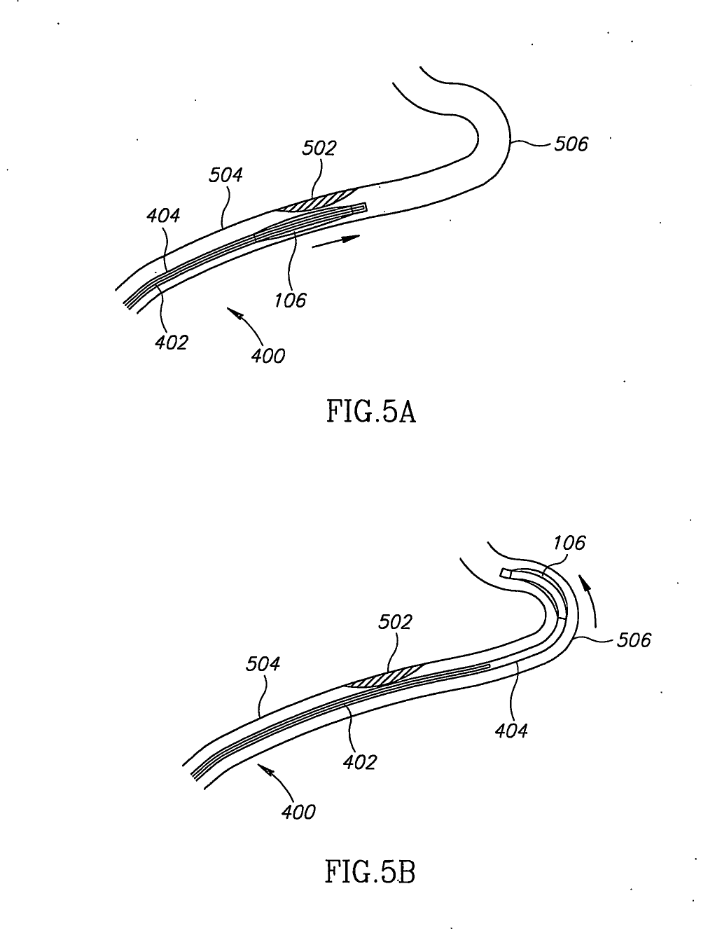

Controlling the flexibility of the tip of a catheter is potentially useful for manipulating the catheter through a blood vessel, as shown in FIGS. 5A and 5B. For example, when trying to push the tip of catheter 400 past an obstruction 502 in a relatively straight portion 504 of a blood vessel, as shown in FIG. 5A, a stiff catheter tip may be desirable. A more flexible catheter tip may be desirable when trying to push catheter 400 past a sharp curve 506 in a blood vessel, as shown in FIG. 5B. By moving inner inflation tube 402 relative to outer inflation tube 404, a flexible portion at the end of the catheter may be made any desired length, depending on what is needed at that time.

Optionally, inner inflation tube 404 is not a complete tube, but has, for example, a C-shaped cross-section, at least in the part of its length that is inside outer inflation tube 402. Optionally, no part of inner inflation tube 404 is a complete tube, and the fluid for inflating balloon 106 is pumped not into inner inflation tube 404, but directly into outer inflation tube 402. In this case, “inner inflation tube” would be a misnomer, and it would be more accurate to call inner inflation tube 404 a “stiffening element” for outer inflation tube 402. However, it is potentially advantageous to make inner inflation tube 404 a complete tube, since that will increase its stiffness and its resistance to buckling, for a given material and inner and outer diameter. For similar reasons, it is potentially advantageous to have inner inflation tube 404 fill up almost all of the cross-sectional area of the lumen of outer inflation tube 402. In that case, there will not be much room for fluid to flow through outer inflation tube 402, so it may be advantageous to inflate balloon 106 through inner inflation tube 404, as described.

It should be appreciated that catheter 100 or catheter 300 is also optionally used in a manner similar to catheter 400, to adjust the stiffness of the catheter tip. In the case of catheter 100 or catheter 300, the tip is optionally made more flexible by using inflation tube 102 to pull balloon 106 back some distance inside outer sheath 104, leaving the distal end of outer sheath 104 empty and relatively flexible. When inflation tube 102 is used to push balloon 106 back up to the distal end of outer sheath 104, then the tip of the catheter (i.e. the distal end of outer sheath 104) becomes stiffer. Depending on the relative stiffness of outer sheath 104, balloon 106 and inflation tube 102, pushing balloon 106 some distance beyond the distal end of outer sheath 104, as shown in FIG. 1, results in a catheter tip (now the balloon and the inflation tube without the outer sheath) of intermediate stiffness.

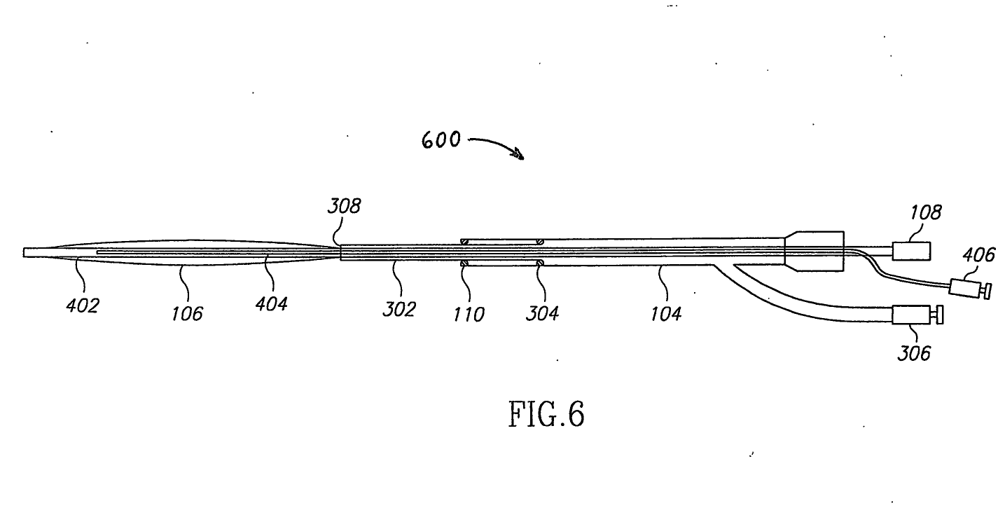

FIG. 6 shows a catheter 600, which combines the features of catheters 300 and 400 of FIGS. 3A and 4A. Catheter 600, like catheter 300, can be fine positioned using any of three different methods, alone or in combination: 1) manipulating the whole catheter; 2) using manipulator 108 to move outer inflation tube 402 (together with balloon 106) relative to outer sheath 104; and 3) using indeflator 306 to move inner sheath 302, together with balloon 106, relative to outer sheath 104. As with catheter 300, any of the hydraulic elements described in PCT/IL03/00995 may be used, instead of or in addition to indeflator 306. Optionally, the hydraulic elements can be configured to look like those in catheter 310 of FIG. 3B, instead of like those in catheter 300 of FIG. 3A.

Like catheter 400, catheter 600 has an inner inflation tube 404, which can be withdrawn back any distance from the tip of the catheter, in order to reduce the stiffness of some portion of the catheter near the tip. The adjustability of the flexibility of catheter 600 is potentially useful both when manipulating the catheter as a whole, and when moving the balloon separately, using either manipulator 108 or indeflator 306, as well as when using any combination of these methods. Thus, catheter 600 potentially provides more options for precise positioning of balloons in difficult situations than catheters 100, 300,310 or 400.

The invention has been described in the context of the best mode for carrying it out. It should be understood that not all features shown in the drawings or described in the associated text may be present in an actual device, in accordance with some embodiments of the invention. Furthermore, variations on the method and apparatus shown are included within the scope of the invention, which is limited only by the claims. Also, features of one embodiment may be provided in conjunction with features of a different embodiment of the invention. As used herein, the terms “have”, “include” and “comprise” or their conjugates mean “including but not limited to.” As used herein, the term “outermost sheath” does not imply that there cannot be still another sheath surrounding it, for example a guide catheter, but only means that the “outermost sheath” is outside the “outer sheath.”

1. A catheter adapted for performing a task at a location inside a lumen, the catheter comprising:
a) an outer sheath;
b) a balloon capable of inflating inside the lumen when the catheter reaches the location; and
c) a balloon inflation tube, which is attached to the balloon and carries a fluid which causes the inflating of the balloon, said balloon inflation tube running through the outer sheath, movable relative to the outer sheath, and stiff enough so that it can be used to push and pull the balloon relative to the outer sheath.

2. A catheter according to claim 1, wherein the inflation tube comprises:
a) a relatively flexible outer balloon inflation tube with a lumen, extending substantially to the tip of the catheter; and
b) a relatively stiff inner inflation tube element, which runs through the lumen of the outer balloon inflation tube and is movable with respect to the outer balloon inflation tube;
whereby moving the inner inflation tube element back from the tip of the catheter makes a distal portion of the catheter substantially more flexible than when the inner inflation tube extends to the tip of the catheter.

3. A catheter according to claim 2, wherein the inner inflation tube element has a lumen which carries the fluid which causes the inflating of the balloon.

4. A catheter according to claim 1, and including a propulsion compartment located proximal to the balloon, the propulsion compartment comprising an outer tube and an inner tube, said tubes being concentric, wherein one of said outer tube and inner tube can slidingly move in relation to the other of said outer tube and inner tube in response to a pressure exerted thereon by a fluid introduced into one or both of said outer tube and inner tube.

5. A catheter according to claim 4, wherein one of said outer tube and inner tube is the outer sheath, and the balloon inflation tube runs through and is attached to the other of said outer tube and inner tube.

6. A catheter according to claim 5, wherein the outer tube is the outer sheath.

7. A catheter according to claim 5, wherein the inner tube is the outer sheath.

8. A catheter adapted for performing a task inside a lumen, the catheter comprising:
a) a balloon capable of inflating inside the lumen; and
b) a balloon inflation tube which is attached to the balloon and carries the fluid which causes the inflating of the balloon, the balloon inflation tube comprising a relative flexible outer balloon inflation tube which extends substantially to the tip of the catheter, and a relatively stiff inner inflation tube element, which runs through the lumen of the outer balloon inflation tube and is movable with respect to the outer balloon inflation tube;
whereby moving the inner inflation tube element back from the tip of the catheter makes a distal portion of the catheter substantially more flexible than when the inner inflation tube extends to the tip of the catheter.

9. A catheter according to claim 8, wherein the inner inflation tube element has a lumen which carries the fluid which causes the inflating of the balloon.

10. A catheter according to claim 1, wherein the task comprises dilating the lumen.

11. A catheter according to claim 1, wherein the lumen is inside the body.

12. A catheter according to claim 11, wherein the lumen is a blood vessel.

13. A catheter according to claim 12, wherein the catheter comprises a stent.

14. A catheter according to claim 1, wherein the balloon inflation tube comprises stainless steel.

15. A catheter according to claim 1, wherein the balloon inflation tube comprises NiTi.

16. A catheter according to claim 1, wherein the balloon comprises plastic.

17. A catheter according to claim 1, wherein the balloon comprises a polymer.

18. A catheter according to claim 1, adapted for using a guide wire.

19. A catheter according to claim 18, adapted for using an “over the wire” guide wire.

20. A catheter according to claim 18, adapted for using a “rapid exchange” guide wire.

21. A method of positioning a balloon of a balloon catheter in a lumen, the method comprising:
a) positioning the balloon approximately; and then
b) fine adjusting the position of the balloon, said fine adjusting comprising moving an inflation tube of the balloon catheter relative to an outer sheath of said catheter, by manually manipulating said inflation tube.

22. A method according to claim 21, wherein moving the inflation tube relative to the outer sheath comprises moving the inflation tube while keeping the outer sheath stationary with respect to the lumen.

23. A method according to claim 21, wherein positioning the balloon approximately comprises moving the entire catheter through the lumen.

24. A method according to claim 23, wherein positioning the balloon approximately also comprises using hydraulic force.

25. A method according to claim 21, wherein positioning the balloon approximately comprises using hydraulic force.

26. A method according to claim 21, wherein fine adjusting also comprises using hydraulic force to move the balloon, while keeping the outer sheath of the catheter stationary with respect to the lumen.

27. A method of manipulating a balloon catheter through a lumen comprising both sharply curved portions and partially obstructed straight portions, the method comprising:
a) arranging a moveable stiffening element to extend substantially to the tip of the catheter, when manipulating the tip of the catheter through the partially obstructed straight portions; and
b) arranging the moveable stiffening element to be withdrawn some distance back from the tip of the catheter, when manipulating the tip of the catheter past the sharply curved portions.

28. A method according to claim 27, wherein the stiffening element is located inside a balloon inflation tube of said catheter.

29. A method according to claim 27, wherein the stiffening element comprises a balloon and a balloon inflation tube of said catheter, and arranging the stiffening element to be withdrawn some distance back comprises withdrawing the balloon into an outer sheath of said catheter.

30. A catheter according to claim 13, wherein the stent is located at substantially the same axial extent of the catheter as the balloon in a configuration suitable for inserting of the catheter into the lumen.

31. A catheter according to claim 13, wherein the stent is adapted to move with the balloon inflation tube when moved relative to the outer sheath.

32. A catheter according to claim 13, wherein moving the balloon inflation tube distally telescopically extends the length of the catheter.
